(19) United States
(12) Patent Application Publication
(10) Pub. No.: US 2004/0193279 A1
(43) Pub. Date: Sep. 30, 2004

(54) Knee posterior stability device

(76) Inventor: Gregory James Roger, Crows Nest (AU)

Correspondence Address: JACOBSON HOLMAN PLLC, 400 SEVENTH STREET N.W., SUITE 600, WASHINGTON, DC, 20004, US

(21) Appl. No.: 10/482,652

(22) PCT Filed: Jul. 10, 2002
(86) PCT No.: PCT/AU02/00948

(30) Foreign Application Priority Data
Jul. 10, 2001 (AU) PR 6263

(51) Int. Cl. A61F 2/38; A61F 2/08

(52) U.S. Cl. 623/20.28; 623/13.12; 623/13.14

(57) ABSTRACT

A device and method that provides stabilisation across a joint of a patient, wherein the device comprising at least one substantially elastic member, the substantially elastic member having a first end and a second end, wherein the first end has a means to allow fixation to a first side of the joint by a first fixation device and the second end has a means to allow fixation to a second side of a joint by a second fixation device.

### TECHNICAL FIELD

The present invention relates to a method and an apparatus for providing posterior support in relation to a total knee replacement.

### BACKGROUND ART

Patients requiring total knee replacement surgery due to joint degeneration often have poor posterior stability due to degeneration of the posterior cruciate ligament. The function of the posterior cruciate ligament can also be impeded due to trauma. During total knee replacement, the posterior cruciate ligament is also often sacrificed due to the geometry of the implant and the poor quality of the ligament.

The posterior cruciate ligament is important for posterior stability of the knee. The posterior cruciate ligament helps maintain effectiveness of the extensor mechanism, providing support during activities such as descending stairs, by driving the tibia forward with respect to the femur and increasing the lever arm about which the patella tendon acts.

One technique used to limit posterior motion in total knee replacement is the provision of a cam post on the polyethylene bearing surface of the tibial component of a knee replacement. The cam post engages with the femoral component of the knee replacement usually between the medial and the lateral condyles of the femoral component in a central cavity. The geometry of the articulating surfaces between the cam post and the femoral component is such that during flexion of the knee, the tibial component is driven forward with respect to the femoral component. This has the effect of limiting posterior motion of the tibial and femoral components, resulting in posterior stability.

Although this technique has proven relatively successful with some patients, several drawbacks and disadvantages are associated with the technique. Often the biomechanical situation created by the cam post is not the condition required for the subject, and the tibia is driven too far forward to the extent that the extensor mechanism subluxes laterally. Due to the requirement of a central cavity in the femoral component, more bone removal is required during surgery to accommodate the cavity within the distal femur, which poses additional clinical considerations and other complications. Loading on the polyethylene cam can also create increased shear stresses within the polyethylene resulting in increased wear debris which in turn can compromise the integrity of the total knee replacement and contribute to aseptic loosening.

In addition, the increased stresses induced in the cam post and the subsequent wear associated with the stresses can result in wear of the cam post to the extent the biomechanics of the system are altered, eventuating in minimal posterior support. Direct translational loading of the polyethylene from the femoral component due to the presence of the cam post also increases shear stresses at the interface between the tibial tray component of the knee replacement and the resected bone, and increases the likelihood of loosening and disassociation between the bone and the implant.

Another technique currently used to provide posterior support in total knee replacement is the provision of a steeper and higher anterior ramp on the lip of the polyethylene liner of the knee replacement. The lip is designed so as to limit posterior motion of the tibial component. This technique has a number of disadvantages associated with it. Before the anterior ramp comes into effect, the tibia must already be at least partially subluxed, hence the extensor mechanism is already at a mechanical disadvantage. Increased stresses at the anterior lip of the polyethylene component results in increased wear and as the polyethylene wears, posterior motion inhibition may be reduced, resulting in poor posterior support.

Another disadvantage of this technique is that the increased ramp only comes into effect when the knee is significantly flexed whereas the effect of a functioning posterior cruciate ligament increases steadily as the knee joint is flexed from full extension, hence the increased ramp does not provide normal biomechanical motion.

The present invention is directed to providing posterior support in conjunction with total knee replacement prostheses in a manner that preferably reduces wear of articular surfaces and provides for a more anatomically normal biomechanical function and motion.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### DISCLOSURE OF INVENTION

The present inventor has recognised the shortcomings of current posterior support devices and practices of total knee replacement prostheses in the prior art and accordingly has sought to provide an improved device and method.

In a first aspect, the present invention is a stabilisation device for providing anterior and posterior support across a knee joint of a patient, the device comprising:

at least one substantially elastic member having a first end and a second end;

the first end having a means to allow fixation to a first side of the joint by a first fixation device and the second end having a means to allow fixation to a second side of a joint by a second fixation device, wherein

a first elastic member is attachable in the anatomical position of an intact anterior cruciate ligament to provide anterior support, and a second elastic member is attachable in the anatomical position of an intact posterior cruciate ligament to provide posterior support;

wherein the stabilisation device is adapted to reduce anterior and posterior translation of the tibia relative to the femur of the patient during flexion or extension of the joint during flexion of the knee joint.

In a second aspect, the present invention is a device that provides posterior stabilisation across a knee joint of a patient in the presence of a knee replacement prosthesis, the device comprising:

a substantially elastic member having a first end and a second end, the first end being attachable to the proximal side of the knee joint, the second end being attachable to the distal side of the knee joint, and wherein the elastic member is adapted to substantially extend in a manner so as to substantially reduce posterior translation of the tibial component relative to the femoral component during flexion of the knee joint,

In a third aspect, the present invention is a knee prosthesis for stabilising a knee joint of a patient, the device comprising:

a femoral component;

a tibial component;

a bearing component; and

a substantially elastic member having a first end and a second end;

wherein, in use, the femoral component is attachable to the distal end of a femur of a patient, the tibial component is attachable to the proximal end of a tibia of the patient, the bearing component is disposable between the femoral component and the tibial component;

and further wherein the first end of the substantially elastic member is attachable to the proximal side of the joint and the second end is attachable to the distal side of the joint and when during flexion of the knee joint, wherein the elastic member is adapted to substantially extend in a manner so as to substantially reduce posterior translation of the tibial component relative to the femoral component.

In a fourth aspect, the present invention is a method of stabilising a knee joint of a patient, the method including the step of:

attaching a first end of an elastic member to a first side of the joint and attaching a second end of an elastic member to a second side of the joint;

wherein the first end has a means to allow fixation to a first side of the joint by a first fixation device and the second end has a means to allow fixation to a second side of a joint by a second fixation device; wherein the elastic member is attached in the anatomical position of an intact anterior cruciate ligament to provide anterior support, or the elastic member is attached in the anatomical position of an intact posterior cruciate ligament to provide posterior support; and

wherein during flexion or extension of the knee joint, the presence of the elastic member reduces anterior or posterior translation of .the tibia relative to the femur of the patient.

In a first embodiment of the first aspect, the elastic member can be made from a material capable of being subjected to relatively large amounts of strain. The material can be synthetic material. Preferably, the material is a hyper-elastic synthetic material. One example of a material used for the elastic member is Nitinol™.

In another embodiment of the first aspect, the elastic member can be used to provide anterior or posterior support across a knee joint of a patient. The first end of the elastic member is attached to the femoral side of the joint and the second end of the elastic member is attached to the tibial side of the joint by means of a fixation device. A screw device or nail device can be used as the fixation device. The elastic member can be fixed in the anatomical position of an intact anterior cruciate ligament (ACL) to provide anterior support, or the elastic member can be fixed in the anatomical position of an intact posterior cruciate ligament (PCL) to provide posterior support.

In a further embodiment of the first aspect, anterior and posterior support can be provided across the knee joint of a patient. A first elastic member can be fixed in the anatomical position of an intact anterior cruciate ligament (ACL) to provide anterior support and a second elastic member can be fixed in the anatomical position of an intact posterior cruciate ligament (PCL) to provide posterior support. To reduce friction and wear between the first elastic member and the second elastic member, bearing material can be attached to the first elastic member and to the second elastic member at a location where the first elastic member and the second elastic member are in contact. Examples of materials that can be used as bearing materials include Teflon™, polyethylene, alumina and zirconia.

In a further embodiment of the first aspect, the device can be secured to the bone of the patient by means of a screw or nail fixation device.

In an embodiment of the second aspect, the knee replacement can be a partial or total knee replacement.

In an embodiment of the second or third aspect, the first end of the elastic member can, in use, be attached to the femoral component of a total knee prosthesis and the second end of the elastic member attached to the tibial component of a total knee prosthesis.

In an alternate embodiment of the second or third aspect, the first end of the elastic member can, in use, be attached to the femoral component of a total knee replacement prosthesis and the second end of the elastic member attached to a proximal portion of the tibia.

In a further embodiment of the second or third aspect, the first end of the elastic member can be attachable to the proximal side of the knee joint by a screw or nail type fixation device, with the fixation device being arranged transverse to the knee joint, in the direction from the lateral side of the knee joint toward the medial side of the knee joint. The first end of the elastic member can further include a bearing means to allow the elastic member to rotate about the longitudinal axis of the nail or screw type fixation device. The bearing device can also further allow the first end of the elastic member to rotate about the radial axis of the screw type fixation device, or “toggle”. This toggling effect can be allowed by sufficient clearance of the bearing means.

In another embodiment of the second or third aspect, the first end of the elastic member is, in use, attached to the proximal side of the joint by means of a transverse member between the femoral condyles of the femoral prosthesis at a location where anatomically a posterior cruciate ligament normally attaches to the femur. The transverse member can be fixedly attached to the femoral prosthesis, or be located between the distal end of the femur and the femoral prosthesis, in a groove introduced surgically prior to implanting the femoral prosthesis. The first end of the elastic member can have a bearing means attachment which allows the elastic member to rotate relatively freely about the axis of the transverse member freely. Examples of materials that can be used for a bearing include polyethylene, Teflon™ and ceramics such as alumina and zirconia. The bearing device can also further allow the first end of the elastic member to rotate about the radial axis of the transverse member or “toggle”. This toggling effect can be allowed by sufficient clearance of the bearing means. The second end of the elastic member is preferably attached to a proximal portion of the tibia at a location where an intact posterior cruciate ligament would be anatomically located by an attachment means. The attachment means can be a securing screw or a means to provide attachment to the posterior portion of the tibial component. A securing screw can be used to anchor the elastic member to the bone. A securing screw similar to screws used in ligament re-attachment procedures can be used. The second end of the elastic member can have a bearing bush attachment at the location at which the member is attached to the securing screw. Examples of materials that can be used for a bearing bush include polyethylene, Teflon™ and ceramics such as alumina and zirconia.

In an embodiment of the fourth aspect, the method is used to stabilise a knee joint of a patient, wherein the first end of the elastic member is attached to the proximal side of the joint and the second end of the elastic member is attached to the distal side of the joint.

In another embodiment of the fourth aspect, the method includes attaching the elastic member in the anatomical position of an intact anterior cruciate ligament to provide anterior support, or the elastic member is attached in the anatomical position of an intact posterior cruciate ligament to provide posterior support. The method can further provide for both anterior and posterior support across a knee joint of a patient, wherein a first elastic member is attached in the anatomical position of an intact anterior cruciate ligament to provide anterior support, and a second elastic member is attached in the anatomical position of an intact posterior cruciate ligament to provide posterior support.

In a further embodiment of the fourth aspect, the method can be used to stabilise a knee joint in the presence of a knee replacement prosthesis, wherein the first end of the elastic member is attachable to the proximal side of the knee joint and the second end is attachable to the distal side of the knee joint such that during flexion of the knee joint posterior translation of the tibial is substantially reduced. The knee replacement prosthesis can be a partial or total knee replacement.

In yet another embodiment of the fourth aspect, the method further comprises the step of replacing the anatomical knee of a patient with a knee replacement prosthesis, the prosthesis comprising a femoral component, a tibial component; and a bearing component, wherein the femoral component is attachable to the distal femur of a patient, the tibial component is attachable to the proximal tibia of the patient, the bearing component is disposable between the femoral component and the tibial component, and the first end of the substantially elastic member is attachable to the proximal side of the joint, the second end is attachable to the distal side of the joint such that during flexion of the joint posterior translation of the tibial is reduced.

In yet a further embodiment of the fourth aspect, the method further comprises the steps of surgically introducing a groove prior to implanting the femoral component, the groove located between the femoral condyles of the knee, and attaching the first end of the elastic member to a transverse member located in the groove, such that when the femoral component is implanted, the transverse member is retained in the introduced groove. Preferably, the first end of the elastic member is attached to the transverse member in a relationship such that the elastic member can rotate about the longitudinal axis of the transverse member during flexion and extension of the knee joint.

In a still further embodiment of the fourth aspect, the first end of the elastic member is attached to the distal femur between the femoral condyles by a fixation device including a screw or nail device, wherein the screw or nail fixation device is arranged transverse to the knee joint in the direction from the lateral side of the knee joint to the medial side of the knee joint. Preferably the first end of the elastic member is attached to the fixation device in a relationship such that the elastic member can rotate about the longitudinal axis of the fixation device during flexion and extension of the knee joint.

In still another embodiment of the fourth aspect, the second fixation device is a bone attachment device including a screw or nail fixation device, wherein the fixation device is integral or separate from the elastic member.

In yet still a further embodiment of the above aspects, the elastic member can have a geometrical shape so as to provide resistance or compliance to physiological loads such that the mechanical function of the elastic member provides correct stabilisation of a joint of a patient. The elastic member can have a geometry which varies in one or more dimensions. Examples of such geometry include a wave-like structure in the direction of the longitudinal axis of the elastic member. The wave-like structure can be planar or can extend in another plane so as to form a spring-like structure. The combination of a suitable material and geometry provides the resistive or compliance properties suitable for providing stability across the joint of a patient, in particularly, to substantially reduce posterior translation of a tibia in the presence of a total knee replacement prosthesis, and provide more anatomically normal biomechanical function and motion. Biomechanical and kinematic motion of a total knee replacement prosthesis varies from that of an intact human knee, and the elastic member, having increased elasticity or compliance due to geometric and material properties in comparison with natural or artificial ligaments, provides for more closer kinematic motion to that required by a total knee prosthesis.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

Examples of the invention are now described with reference to the accompanying drawings:

FIG. 1—examples of elastic member configurations;

FIG. 2—examples of an elastic member providing support across the knee joint of a patient;

FIG. 3—sectional view of elastic member and knee prosthesis;

FIG. 4—tibial component of knee prosthesis; and

FIG. 5—femoral component of knee prosthesis

### DETAILED DESCRIPTION OF THE DRAWINGS

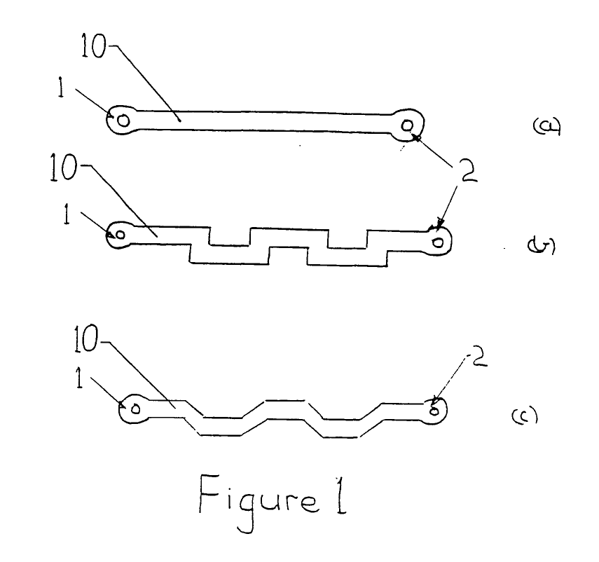

FIG. 1 shows examples of configurations of the elastic member 10. The elastic member 10 has a first end 1 and a second end 2. The elastic member 10 can be made of a hyper-elastic material or can be made of a super elastic material. An example of a material that the elastic member can be made of is Nitinol™, a super-elastic shape memory alloy.

The elastic member 10 can also be made of polymeric materials or reinforced polymeric materials. The geometry of the elastic member 10 can be such that appropriate behavioural characteristics are achieved so as when used to provide stability across a joint of a patient, appropriate stability is obtained. The elastic member 10 can have a varying geometry in one or more directions.

The elastic member can have a geometry which varies in one or more dimensions. Examples of such geometry include a wave-like structure in the direction of the longitudinal axis of the elastic member. The wave-like structure can be planar or can extend in another plane so as to form a spring-like structure. The combination of a suitable material and geometry provides the resistive or compliance properties suitable for providing stability across the joint of a patient.

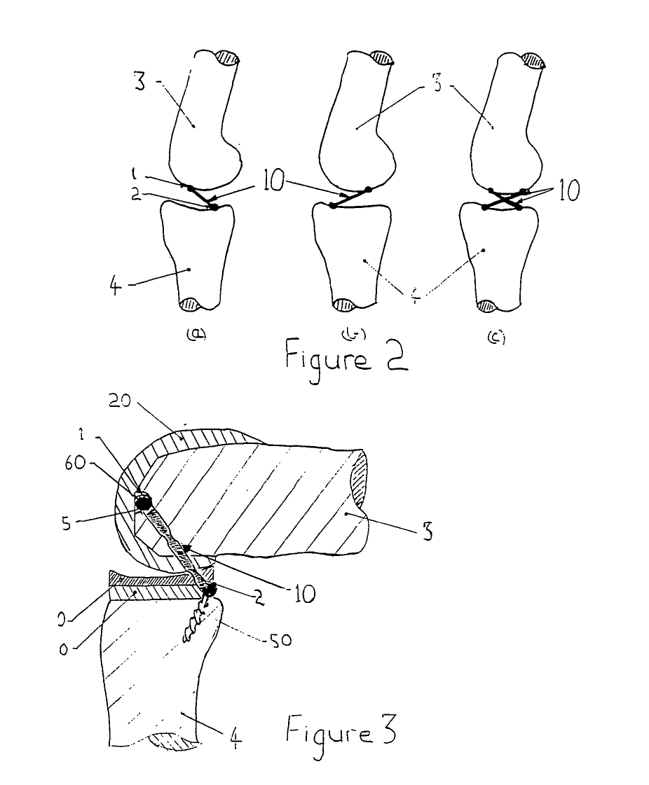

FIG. 2 shows examples of the elastic member 10 being used to provide stability across a knee joint of a patient.

In FIG. 2 (a), the elastic member 10 is used to provide posterior support of a knee joint. The first end of the elastic member 1 is fixed to an anterior location on the distal femur 3 and the second end 2 of the elastic member 10 is fixed to a posterior location on the proximal tibia 4. The elastic member can be located approximately as would an intact posterior cruciate ligament (PCL) of a patient be located.

In FIG. 2 (b), the elastic member 10 is used to provide anterior support of a knee joint. The first end of the elastic member 10 is fixed to a posterior location on the distal femur 3 and the second end 2 of the elastic member 10 is fixed to an anterior location on the proximal tibia 4. The elastic member can be located approximately as would an intact anterior cruciate ligament (ACL) of a patient be located.

FIG. 2 (c) shows a first and a second elastic member 10 being used to provide both posterior and anterior support of a knee joint of a patient. The first end of a first elastic member 1 is fixed to an anterior location on the distal femur 3 and the second end 2 of the elastic member 10 is fixed to a posterior location on the proximal tibia 4. The elastic member can be located approximately as would an intact posterior cruciate ligament (PCL) of a patient be located. The first end of a second elastic member 10 is fixed to a posterior location on the distal femur 3 and the second end 2 of the elastic member 10 is fixed to an anterior location on the proximal tibia 4. The elastic member can be located approximately as would an intact anterior cruciate ligament (ACL) of a patient be located. In the region where the first elastic member 10 and the second elastic member impinge upon each other, bearing bushes can be used to reduce wear. Suitable materials for bearing bushes include polyethylene, Teflon™, alumina and zirconia.

FIG. 3 shows a sectional view of the use of the elastic member 10 in conjunction with a knee prosthesis to provide posterior support and stability. A femoral component 20 is surgically fixed to the distal femur 3 of a patient. The tibial component 30 is surgically fixed to the proximal tibia 4 of a patient. The bearing component 40 can be attached to the tibial component 30. In a first example, the elastic member 10 is separate from the components of the knee prosthesis. The figure depicts the knee in a flexed position at which the elastic member 10 is in a stretched and stressed state. As is shown, when the knee is flexed, the elastic properties of the elastic member 10 maintain the tibia 4 of the patient such that posterior translation of the tibia 4 with respect to the femur 3 is reduced. As illustrated, the femoral component 20 is not riding on the anterior lip of the bearing component 40 as is the case when the elastic member 10 is not present.

When the knee is extended, the elastic properties of the elastic member 10 cause the elastic member 10 to reduce in length such that it does not interfere or impinge upon component or structures of the knee. The elastic member 10 can be provided with a limit means such that the elastic member bears substantially no compressive load. Examples of such limit means include a partially collapsable portion or not-loading bearing. Alternatively, the elastic member can have compressive properties such that at least partial resistance to anterior translocation of the tibia 4.

Prior to fixation of the femoral component, a small groove 5 can be made on the distal face of the distal femur 3 to house an axle 60. The axle 60 can be attached to the first end 2 of the elastic member 10. The axle 60 can be made from materials including stainless steel, cobalt-chrome and titanium alloy. A bearing bush can be used at the interface of the axle 60 and the elastic member 10 to allow rotation and reduce wear. Suitable materials for the bearing bush include polyethylene, Teflon™, alumina and zirconia. The femoral component 20 when fixed to the distal femur, maintains the axle 60 in the groove 5, which is attached to the elastic member 10, so that the elastic member 10 is effectively fixed to the distal femur 3. In this example, the second end 2 of the elastic member is attached directly to the bone of the proximal tibia 4 by means of a screw device 50. A bearing device can be present between the second end of the elastic member 2 and the screw device. Suitable materials for the bearing include polyethylene, Teflon™, alumina and zirconia.

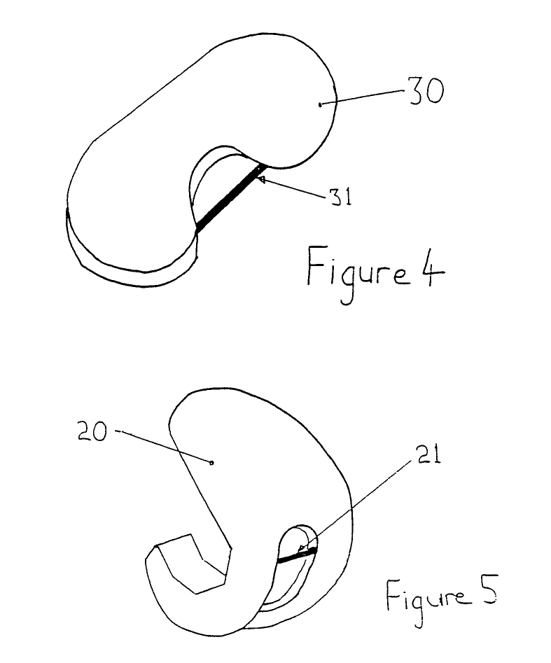

FIG. 4 shows an example of a tibial component 30 used in an embodiment of the invention in which the second end 2 of the elastic member 10 is attached effectively to the tibial component 30. The second end 2 of the elastic member 10 is attached to a bar member 31 located between the posterior lobes of the tibial component 30. The bar member 31 can be integral or separate from the tibial component 30. A bearing device can be present between the second end 2 of the elastic member 10 and the bar member 31. Suitable materials for the bearing include polyethylene, Teflon™, alumina and zirconia.

FIG. 5 shows an example of a femoral component 20 used in an embodiment of the invention in which the first end 1 of the elastic member 10 is attached effectively to the femoral component 20. The first end 1 of the elastic member 10 is attached to a connection member 21 located between the condyles of the femoral component 20. The connection member 21 can be integral or separate from the femoral component 20. A bearing device can be present between the first end 1 of the elastic member 10 and the connection member 21. Suitable materials for the bearing include polyethylene, Teflon™, alumina and zirconia.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-36. (canceled)

37: A stabilisation device for providing anterior and posterior support across a knee joint of a patient, the device comprising:
at least one substantially elastic member having a first end and a second end;
the first end having a means to allow fixation to a first side of the joint by a first fixation device and the second end having a means to allow fixation to a second side of a joint by a second fixation device, wherein a first elastic member is attachable in the anatomical position of an intact anterior cruciate ligament to provide anterior support, and a second elastic member is attachable in the anatomical position of an intact posterior cruciate ligament to provide posterior support;
wherein the stabilisation device is adapted to reduce anterior and posterior translation of the tibia relative to the femur of the patient during flexion or extension of the joint during flexion of the knee joint.

38: The device according to claim 37, further comprising a bearing member attached to the first elastic member and/or the second elastic member at a location where the first elastic member and the second elastic member are in contact;
wherein the bearing member reduces friction and wear between the first elastic member and the second elastic member.

39: The device according to claim 38, wherein the bearing member is made of a biocompatible material including polytetrafluoroethylene, polyethylene, alumina and zirconia.

40: A device according to claim 39, wherein the first fixation device and/or the second fixation device is a bone attachment device including a screw or nail fixation device;
wherein the fixation device is integral or separate from the elastic member.

41: A device that provides posterior stabilisation across a knee joint of a patient in the presence of a knee replacement prosthesis, the device comprising:
a substantially elastic member having a first end and a second end, the first end being attachable to the proximal side of the knee joint, the second end being attachable to the distal side of the knee joint, and wherein the elastic member is adapted to substantially extend in a manner so as to substantially reduce posterior translation of the tibial component relative to the femoral component during flexion of the knee joint,

42: A knee prosthesis for stabilising a knee joint of a patient, the device comprising:
a femoral component;
a tibial component;
a bearing component; and
a substantially elastic member having a first end and a second end;
wherein, in use, the femoral component is attachable to the distal end of a femur of a patient, the tibial component is attachable to the proximal end of a tibia of the patient, the bearing component is disposable between the femoral component and the tibial component;
and further wherein the first end of the substantially elastic member is attachable to the proximal side of the joint and the second end is attachable to the distal side of the joint and when during flexion of the knee joint, wherein the elastic member is adapted to substantially extend in a manner so as to substantially reduce posterior translation of the tibial component relative to the femoral component.

43: The device according to claim 42, wherein the knee joint is a partial or a total knee replacement.

44: The device according to claim 42, wherein the second end of the elastic member is attachable to the distal side of the knee joint by a fixation means, the fixation means including a screw or nail fixation device;
wherein the fixation means is integral or separate from the elastic member.

45: The device according to claim 44, wherein the first end of the elastic member is attachable to the proximal side of the knee joint by a screw or nail fixation device, wherein the screw or nail fixation device is arranged transverse to the knee joint, in the direction from the lateral side of the knee joint to the medial side of the knee joint.

46: The device according to claim 45, wherein the first end of the elastic member further includes a bearing means to allow the elastic member to rotate about the longitudinal axis of the fixation device;
wherein the bearing means is formed from a biocompatible material including polyethylene, polytetrafluoroethylene, alumina and zirconia.

47: The device according to claim 46, wherein the bearing means further allows the elastic member to at least partially rotate about the radial axis of the fixation device.

48: The device according to claim 45, wherein the first end of the elastic member is attachable to the proximal side of the knee joint by attachment to a transverse member;
wherein the transverse member is integral with the femoral component of the knee joint prosthesis, located between the femoral condyles of the knee and is attached to at least one femoral condyle of the femoral component of the knee prosthesis.

49: The device according to claim 44, wherein the first end of the elastic member is attachable to the proximal side of the knee joint by attachment to a transverse member;
wherein the transverse member is locatable between the distal end of the femur and the femoral prosthesis and locatable in a groove introduced surgically prior to implanting the femoral component, the groove located between the femoral condyles of the knee.

50: The device according to claim 49, wherein the first end of the elastic member further includes a bearing means to allow the elastic member to rotate about the longitudinal axis of the transverse member;
wherein the bearing means is formed from a biocompatible material including polyethylene, polytetrafluoroethylene, alumina and zirconia.

51: The device according to claim 50, wherein the bearing means further allows the elastic member to at least partially rotate about the radial axis of the transverse member.

52: The device according to claim 51, wherein the bearing means allows the elastic member to at least partially rotate about the radial axis of the transverse member by sufficient clearance between the bearing member and the transverse member.

53: The device according to claim 42, wherein the elastic member is formed from a material suitable for being subjected to amounts of strain so as to provide stability of the knee joint throughout normal angles of flexion and extension.

54: The device according to claim 53, wherein the material is a synthetic material, including hyper-elastic materials.

55: The device according to claim 54, wherein the synthetic material is Nitinol™.

56: The elastic member according to claim 42, wherein the elastic member has a geometrical shape so as to provide resistance or compliance to physiological loads such that the mechanical function of the elastic member provides correct stabilisation of the joint of the patient.

57: The elastic member according to claim 42, further comprising a bearing at the second end of the elastic member to allow the elastic member to freely rotate when secured to bone by a fastening means.

58: The elastic member according to claim 42, wherein the elastic member provides at least partial resistance to anterior translocation of the tibia of the patient.

59: The elastic member according to claim 58, wherein the geometrical and material properties of the elastic member in combination provide at least partial resistance to anterior translocation of the tibia of the patient.

60: The elastic member according to claim 42, wherein the elastic member is further provided with a limit means to allow the elastic member to bear substantially no compressive load.

61: The elastic member according to claim 60, wherein the limit means is a hinged portion located between the first end and the second end of the elastic member.

62: The elastic member according to claim 60, wherein the limit means includes an ovoid aperture at at least one end of the elastic member;
wherein under tensile load, an internal surface of the ovoid aperture bears against a portion of a fixation device, and wherein under compressive load, the internal surface of the ovoid aperture does not transfer load to the fixation device.

63: A method of stabilising a knee joint of a patient, the method including the step of:
attaching a first end of an elastic member to a first side of the joint and attaching a second end of an elastic member to a second side of the joint;
wherein the first end has a means to allow fixation to a first side of the joint by a first fixation device and the second end has a means to allow fixation to a second side of a joint by a second fixation device; wherein the elastic member is attached in the anatomical position of an intact anterior cruciate ligament to provide anterior support, or the elastic member is attached in the anatomical position of an intact posterior cruciate ligament to provide posterior support; and
wherein during flexion or extension of the knee joint, the presence of the elastic member reduces anterior or posterior translation of the tibia relative to the femur of the patient.

64: The method according to claim 63 for providing anterior and posterior support across a knee joint of a patient, wherein a first elastic member is attached in the anatomical position of an intact anterior cruciate ligament to provide anterior support, and a second elastic member is attached in the anatomical position of an intact posterior cruciate ligament to provide posterior support;
wherein during flexion or extension of the knee joint, the presence of the elastic member reduces anterior and posterior translation of the tibia relative to the femur of the patient.

65: The method of claim 63, wherein the knee joint includes a knee replacement prosthesis;
wherein the first end of the elastic member is attachable to the proximal side of the knee joint and the second end is attachable to the distal side of the knee joint;
and during flexion of the knee joint, the presence of the elastic member reduces posterior translation of the tibia relative to the femur of the patient.

66: The method according to claim 65, wherein the knee replacement prosthesis is a partial or total knee replacement.

67: The method according to claim 63, wherein the joint is a knee joint, the method further including the step of:
replacing the anatomical knee of a patient with a knee replacement prosthesis, the prosthesis comprising:
a femoral component;
a tibial component; and
a bearing component;
wherein the femoral component is attachable to the distal femur of a patient, the tibial component is attachable to the proximal tibia of the patient, the bearing component is disposable between the femoral component and the tibial component, and the first end of the substantially elastic member is attached to the proximal side of the joint, the second end is attached to the distal side of the joint, and during flexion of the knee joint, and;
wherein the elastic member is adapted to substantially extend in a manner so as to substantially reduce posterior translation of the tibial component relative to the femoral component.

68: The method according to claim 67, further including the steps of:
surgically introducing a groove prior to implanting the femoral component, the groove located between the femoral condyles of the knee; and
attaching the first end of the elastic member to a transverse member located in the groove, such that when the femoral component is implanted, the transverse member is retained in the introduced groove.

69: The method according to claim 68, wherein the first end of the elastic member is attached to the transverse member in a relationship such that the elastic member can rotate about the longitudinal axis of the transverse member during flexion and extension of the knee joint.

70: The method according to claim 67, wherein the first end of the elastic member is attached to the distal femur between the femoral condyles by a fixation device including a screw or nail device;
wherein the screw or nail fixation device is arranged transverse to the knee joint in the direction from the lateral side of the knee joint to the medial side of the knee joint.

71: The method according to claim 70, wherein the first end of the elastic member is attached to the fixation device in a relationship such that the elastic member can rotate about the longitudinal axis of the fixation device during flexion and extension of the knee joint.

72: The method according to claim 69, wherein the second fixation device is a bone attachment device including a screw or nail fixation device;
wherein the fixation device is integral or separate from the elastic member.
